(19) United States
(12) Patent Application Publication
(10) Pub. No.: US 2015/0352777 A1
(43) Pub. Date: Dec. 10, 2015

(54) HIGH-THROUGHPUT MANUFACTURING OF MICRONEEDLES

(71) Applicants: THE UNIVERSITY OF NORTH CAROLINA AT CHAPEL HILL, Chapel Hill, NC (US); LIQUIDIA TECHNOLOGIES, INC., Durham, NC (US)

(72) Inventors: Joseph DeSimone, Chapel Hill, NC (US); Michael Hunter, Cary, NC (US); Katherine Anne Moga, Stow, OH (US)

(21) Appl. No.: 14/761,651

(22) PCT Filed: Jan. 17, 2014
(86) PCT No.: PCT/US14/12059

§ 371 Date: Jul. 17, 2015

Related U.S. Application Data
(60) Provisional application No. 61/754,116, filed on Jan. 18, 2013.

(51) Int. Cl. B29C 59/00; A61M 37/00

(52) U.S. Cl. CPC B29C 59/002; A61M 37/0015; B29K 2033/04

(57) ABSTRACT

The present invention provides a system for fabricating a microneedle device and microneedle devices. The system includes depositing a first material on a delivery sheet, introducing the delivery sheet with deposited first material to a mold, passing the delivery sheet with deposited first material and mold through a nip-point to introduce at least a portion of first material into cavities in the mold, and separating the mold from the first material to provide a microneedle device with needles mimicking the cavities of the mold.

### FEDERALLY SPONSORED RESEARCH OR DEVELOPMENT

This invention was made with government support under Grant No. CA151652 awarded by the National Institutes of Health. The government has certain rights in the invention.

### TECHNICAL FIELD OF THE INVENTION

In general the present invention relates to the field of drug delivery through microneedles. More particularly, the present invention relates to the high-throughput manufacture of microneedle arrays.

### BACKGROUND

Microneedle arrays of micron-sized projections can painlessly pierce the epidermis and deliver therapy to the skin. While microneedles have been made from a variety of materials and configurations, current biodegradable microneedle devices require slow, extensive processing with inherent manufacturing limitations that are overcome by the present invention. Current fabrication of biodegradable microneedle devices utilize batch-processing with many individual time consuming stages. The present invention provides efficient roll-to-roll manufacturing of microneedle devices in a continuous linear fashion.

### SUMMARY OF THE INVENTION

The present invention provides a system for fabricating a microneedle device. The system includes depositing a first material on a delivery sheet, introducing the delivery sheet with deposited first material to a mold, passing the delivery sheet with deposited first material and mold through a nip-point to introduce at least a portion of first material into cavities in the mold, and separating the mold from the first material to provide a microneedle device with needles mimicking the cavities of the mold, as described more fully herein.

### BRIEF DESCRIPTION OF THE FIGURES

FIGS. 1A-1D shows manufacturing of microneedle devices according to an embodiment of the present invention;

FIGS. 2A-2C shows manufacturing of multi-component microneedle devices according to an embodiment of the present invention;

FIGS. 3A and 3B show microneedle devices according to an embodiment of the present invention;

FIG. 4 shows a system for continuous manufacturing of microneedle devices of the present invention;

FIG. 5A-5B shows a schematic of the application of the biodegradable microneedles of the present invention where the needles and substrate are inserted into the skin of a patient. FIG. 5C shows the backing of the microneedle patch dissolved with a solvent;

FIG. 6 shows a “mother-ship” microneedle (400 μm tall), an embodiment of the present invention, with fluorescently tagged 1 μm BSA PRINT particles dispersed throughout needle;

FIGS. 7A-7C shows making a microneedle master template through an inclined and rotated photolithography schematic. FIG. 7A shows an SU-8 coated wafer placed on a tilted stage (18-25°) and exposed; FIG. 7B shows the substrate rotated 90° about the surface normal and exposed once more; and FIG. 7C shows, after a total of four exposures, the wafer is post-exposure baked (PEB) and developed, leaving a negative master template;

FIGS. 8A and 8B show an ESEM image of SU-8 Master template; FIGS. 8C and 8D show a PDMS template; FIGS. 8E and 8F show a PFPE mold; and FIGS. 8G and 8H show PVP based microneedles made from R2 SU-8 master having 200 μm squares with 200 μm spacing according to an embodiment of the present invention. Needles show comparable lengths and tip diameters. Scale bars on FIGS. 8 A, C, E, and G are 500 μm and scale bars for FIGS. 8 B, D, F, and H are 200 μm;

FIGS. 9A and 9B show a schematic of the process for making microneedles, including the fabrication of individual microneedles and harvesting onto a flexible, water-soluble substrate according to an embodiment of the present invention;

FIG. 10A-10C show brightfield macroscopic images of a microneedle patch; including the microneedle array morphology showing large area, reproducible, uniform microneedles according to an embodiment of the present invention. Scale bar is 200 μm;

FIG. 11 shows brightfield macroscopic image of ex vivo murine skin after testing with microneedle patch of the present invention for 10 seconds. The pattern of the microneedles can be seen on the skin and shown in the insert is a single piercing;

FIG. 12A-12C show brightfield microscopy images of skin penetration studies performed on ex vivo murine skin with microneedles of the present invention. FIG. 12A shows control skin; FIG. 12B shows skin after 10 second microneedle application; and FIG. 12C shows skin after 10 minute microneedle application with microneedles of the present invention. Scale bar on all FIG. 12 images is 35 μm;

FIG. 13A-13C shows fluorescent microscopy images of skin penetration studies performed on ex vivo murine skin with microneedles of the present invention. FIG. 13A shows control skin; 13B shows skin after 10 second microneedle application; and 13C shows skin after 10 minute microneedle application with microneedles of the present invention. Scale bar on all FIG. 13 images is 35 μm;

FIG. 14 shows microscopy images of skin penetration studies performed on ex vivo human skin from a patient with IBC using microneedles of the present invention;

FIG. 15 shows microneedle patches after insertion into skin. In FIG. 15A, microneedle patch was inserted for 10 seconds and a brightfield image of the skin and in FIG. 15B, microneedle patch was inserted for 10 minutes and a fluorescent image of the skin was taken. FIG. 15 uses a common scale bar of 70 μm;

FIG. 16 shows chemical structures of PEG diacrylate with n chains (A), PEG dimethacrylate with n chains (B), and DMAEM (C);

FIG. 17 shows ESEM images of microneedles comprised mainly of PEG diacrylate with molecular weight 700. 17(A) General overview of microneedle array. 17(B) Image of microneedle tip with a tip radius of curvature of 11.45 μm, small enough to generate enough insertion force to pierce skin. 17(C) Image shows the uniformity of the tips in a microneedle array. 17(D) Image shows a height of 381.23 μm, a height tall enough for to pierce stratum corneum;

FIG. 18 shows microneedles of each hydrogel matrix (see Table 1) before and (left) and after (right) swelling. (Top) Comparison of “short” chain matrices, and (Bottom) comparison of “long” chain matrices. Swelling in the “long” chain matrices showed needles that maintained their shape at 60% relative humidity while still expanding. Scale bar in all images is 200 μm; and

FIG. 19 shows brightfield microscopic images of cryosectioned mouse skin tissue. (A) Control skin (no microneedles applies); (B) Film control (hydrogel film applied for 10 minutes); (C) Skin treated with PEG575diacrylate microneedles, showing epidermal breach, indicated by the arrow, and (D) Skin treated with PEG550dimethacrylate showing epidermal breach, indicated by the arrow.

### DETAILED DESCRIPTION

The present invention overcomes the barriers in the fabrication of microneedles reported in the literature by providing sheet based molding techniques, systems and methods described herein. Generally, the process begins with a pre-microneedle solution 100 that can contain a desired composition, including but not limited to a host of matrices including polymers, monomers, drugs, nucleic acids, or any additional agent (i.e., active agent) of interest. The pre-microneedle solution is then deposited onto a delivery sheet 105, forming a thin-film first layer 100. Next, the delivery sheet 105/first layer 100 combination is processed through a nip point 102 with a mold 120 to introduce the material of first layer into cavities 125 in mold 120. Thereafter, the mold is removed leaving microneedle device with needles 150 that mimic the cavities in the mold and a foot-print size that mimics the size of the mold.

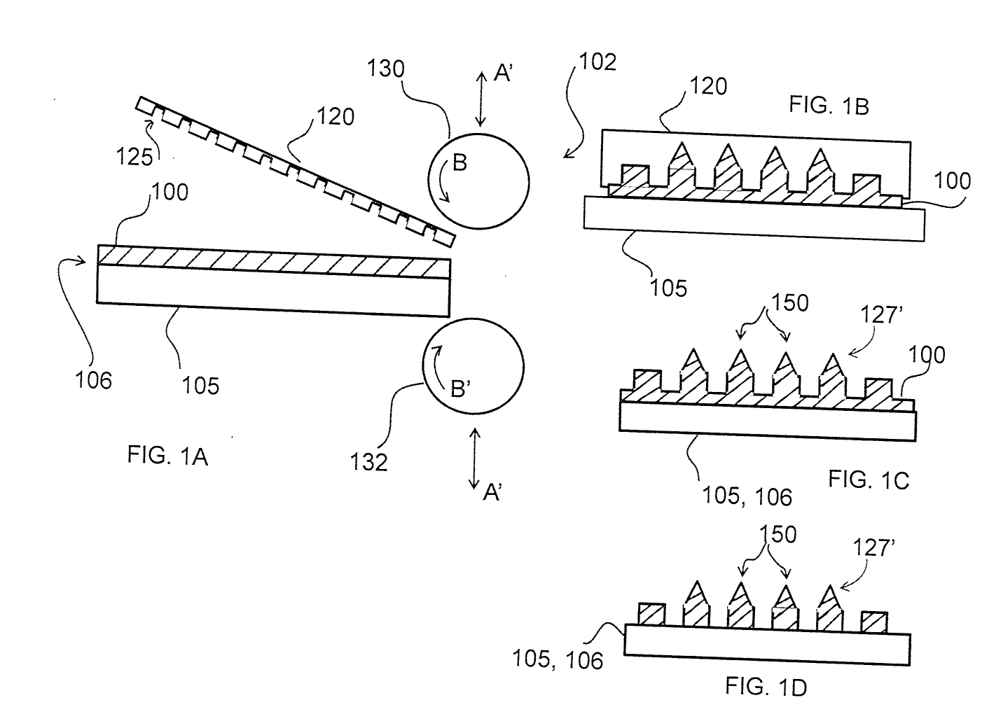

FIG. 1A shows an embodiment for high throughput thin-film manufacturing equipment, methods and systems for manufacturing microneedle arrays. According to such embodiment, delivery sheet 105 can be, for example a thin film of PET or other suitable material as will be appreciated by one of ordinary skill in the art. In some embodiments, delivery sheet 105 can be a sheet that is biocompatible, bioresorbable, or the like and also used as the backing sheet for applying the microneedles to a patient. In an alternative embodiment, an application layer 106 can be included onto delivery sheet 105 before depositing first layer 100 on delivery sheet 105. According to such embodiments, application layer 106 can be an adhesive layer, a bioresorbable, water (or other solvent) soluble layer, or the like. During fabrication, first layer 100 is applied to application layer 106, which later is utilized to handle and apply the microneedle device to a patient. In an alternative embodiment, delivery sheet 105 is a continuous thin film used in a roll-to-roll processing system of the present invention, which can provide microneedles of the present invention in continuous length webs, such as for example, up to 24 inches wide or more and up to 5000 feet in length or more.

In one embodiment, a first layer 100 of material, which will form the base and needle portion of the microneedle device 300, is deposited on the delivery sheet 105. First layer of material 100 can be deposited on delivery sheet 105 by any technique in the art, such as for example, spraying, depositing droplets, pouring, coating, or the like. In some embodiments, it is important to deposit first layer 100 in a controlled manner such that the material of first layer 100 is a thin film having a highly consistent thickness across delivery sheet 105 in both width and length dimensions.

The delivery sheet 105, with first layer 100 deposited thereon, is brought into contact with mold 120 through a nip roller 102. Nip roller 102 consists of two rollers 130,132 that are rotatable in direction of arrows B,B′ and can be configured with a fixed separation distance or can be movable in direction of arrows A,A′. Nip roller 102 forms a nip-point along a line connecting the center-line of rollers 130,132. In an alternative embodiment, nip roller 102 consists of a single roller 130 and a contact surface rather than a second roller. In such embodiment, nip roller 102 forms a nip point along a line between the center-line of roller 130 and the base surface it contacts. Either of the rollers 130,132 of nip roller 102 can be, in some embodiments, heated, driven or non-driven, pressure controlled or fixed pressure, metal surfaces, rubber surfaces, or the like.

An important aspect of the present invention includes the use of nip roller 102 to form microneedles of the present invention. Using a nip roller 102 forms a point of linear contact extending along axis of rollers 130,132 which makes conformal contact between mold and delivery sheet 105, thereby helping first layer 100 into cavities of mold. Moreover, as rollers 130, 132 or nip roller 102 are rotated and the combined delivery sheet/mold pass through nip roller 102, the linear conformal contact along the line of roller 130,132 axis is translated into sheet conformal contact as the material of first layer 100 is brought into contact with mold and mold cavities. For a given set of materials of first layer 100, such as for example; flowability, glass transition temperature, melting temperature, cooling rate, crystallinity, modulus, and the like, different conditions of the system, such as for example, roller temperature, line speed, line width, cavity depth, cavity size, air trapping/removal from cavities, and the like, will be required to flow, fill, and form the microneedles of the present invention.

When first material 100 encounters the mold 120 in the nip point formed between rollers 130,132, first material 100 fills cavities 125 in mold 120, which ultimately forms the needle portions 150 of microneedles after first material 100 is removed from mold 120, as shown in FIGS. 1B-D. In some embodiment, first material 100 is a flowable solid, such as for example a flowable power or granules, or a liquid (or semi-liquid) such that first material 100 flows into mold cavities 125. In alternative embodiments, first material 100 is heated at nip-point by use of a heated roller 130,132 and thereafter flows into mold cavities 125. In yet other embodiment, first material 100 is drawn into cavities 125 through capillary force or other such forces exerted on first material 100 through laminate contact between the top surface of first material 100 and mold 120, as shown in FIG. 1B. As shown in FIG. 1D, in some embodiments first material 100 is metered onto delivery sheet 105 to control the volume and/or thickness of first material 100 deposited such that all or substantially all of first material 100 is utilized when first material 100 enters cavities 125, thereby forming needles 150 of microneedle array on a backing layer 105,106 without or substantially without an interconnecting layer of first material 100 extending between adjacent needles 150.

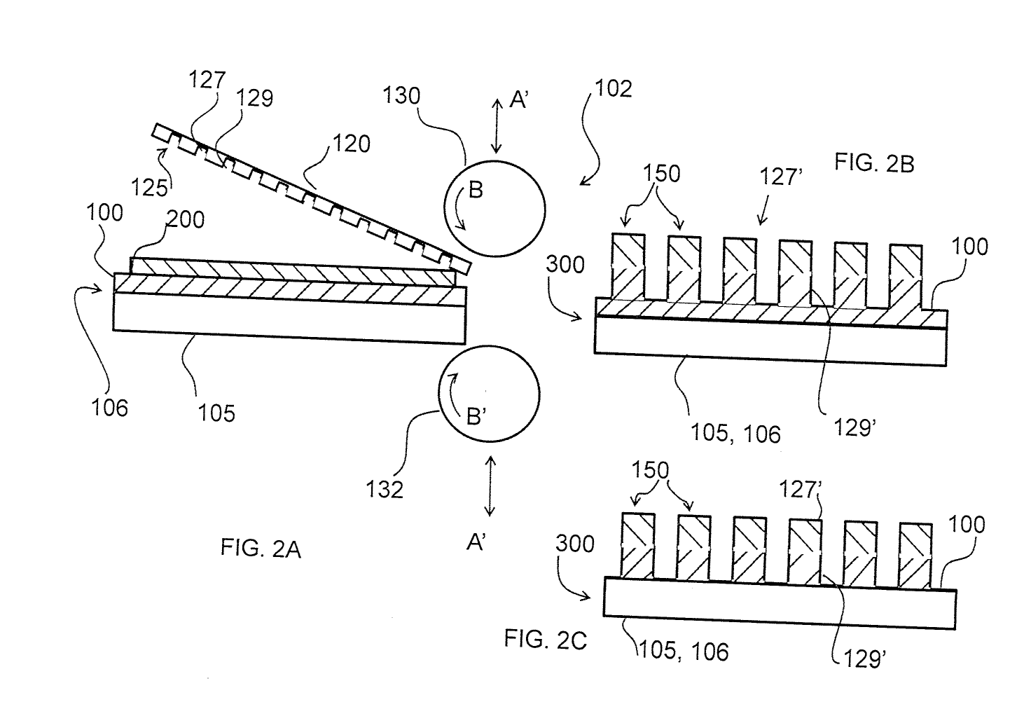

As shown in FIG. 2A and according to another embodiment of the present invention a second later 200 is deposited onto first layer 100 prior to first layer 100 being brought into contact with mold 120 in nip roller 102. In such embodiments, second layer can include an active ingredient, such as for example a pharmaceutical agent, biologic drug, charged molecule for scavenging molecules from an in-vivo location, or the like. Second layer 200 can be deposited by the same or different approach as first layer 100 (described herein). In some embodiments, controlling the thickness and/or uniformity of second layer 200 is essential to producing the resulting needles 150 of microneedle device with a consistent and uniform active agent loading between the individual needles of the device. Accordingly, in some embodiments, second later 200 is deposited on first layer 100 in a thin film having a thickness less than the depth of cavities 125. According to such embodiments, the deposited thickness of second layer 200 on first layer 100 can be adjusted to provide the calculated active agent loading into needles 150 of microneedle device as needed for a particular treatment regime. Factors included in the calculation of needle loading include, potency and dosing of active agent of choice, the number of needles included on device, the volume, shape, and size of the needles, the depth of tissue penetration desired for the given application, and the like.

As first layer 100 and second layer 200 deposited on the delivery sheet 105 passes through nip point formed between rollers 130,132 of nip roller 102, second material 200 enters the cavities 125 of mold 120 before first layer 100 enters the cavities. According to some embodiments, the thickness of deposited second layer 200 is tailored to provide that second layer 200 only fill the tip-most region 127 of the cavities 125 in mold while first material 100 forms the bulk of the needle 129 by filling the remainder of the cavity 125 in mold 120. In such embodiment, second material 200 can deliver the active ingredient while first material 100 can give microneedle the necessary mechanical support for, for example, piercing a biologic barrier for delivery of the active ingredient to a patient. In alternative embodiments, as shown in FIG. 2B, the deposited thickness of both first layer 100 and second layer 200 can be tailored such that cavities 125 of mold 120 consumes all or substantially all of the volume of deposited first layer 100 and second layer 200 and forms needles 150 with no or substantially no interconnection between respective needles 150. As a result, microneedle device 300 includes needles 150 with tip regions 127′ and base regions 129′, respectively formed from the material of second layer 200 and first layer 100. In an alternative embodiment, controlled deposition of the volume and/or thickness of second layer 200 to provide an equivalent volume of second layer 200 than the collective volume of cavities 125 in mold 120 results in needles 150 formed from the material of the second layer 200 with no or substantially no interconnection between respective needles 150. In another alternative embodiment, controlled deposition of the volume of second layer 200 to provide a volume of second layer 200 that is greater than 90 percent of the collective volume of cavities 125 in mold 120 results in needles 150 formed from the material of the second layer 200 with no or substantially no interconnection between respective needles 150.

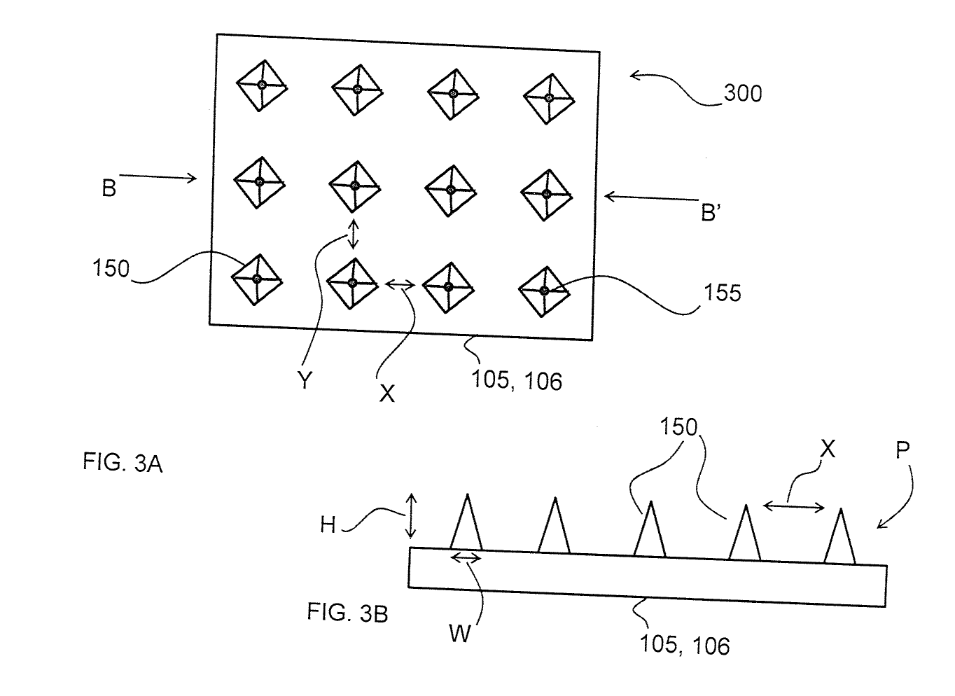

FIG. 3A shows a top plan view of microneedle array 300 of the present invention. As shown, needles 150 of microneedle array 300 can be coordinated in an array pattern and spacing in the X and Y directions can be controlled based on spacing of cavities 125 in mold 120. Furthermore, needles 150 have a tip 155 that results from the deepest portion of cavities 125 in mold 120. Each needle 150 also has a profile P, width W, and height H (see FIG. 3B which is a cross-section taken along the line B-B′ of FIG. 3A) that results from needle 150 mimicking the three-dimensional shape of cavities 125 in mold 120. Accordingly, as will be appreciated by one of ordinary skill in the art, parameters X, Y, W, H, and P can be adjusted, as appropriate, for the delivery of respective active agents, delivery site, pain or pain-free application of the device to a patient, mechanical properties of needles 150, and the like.

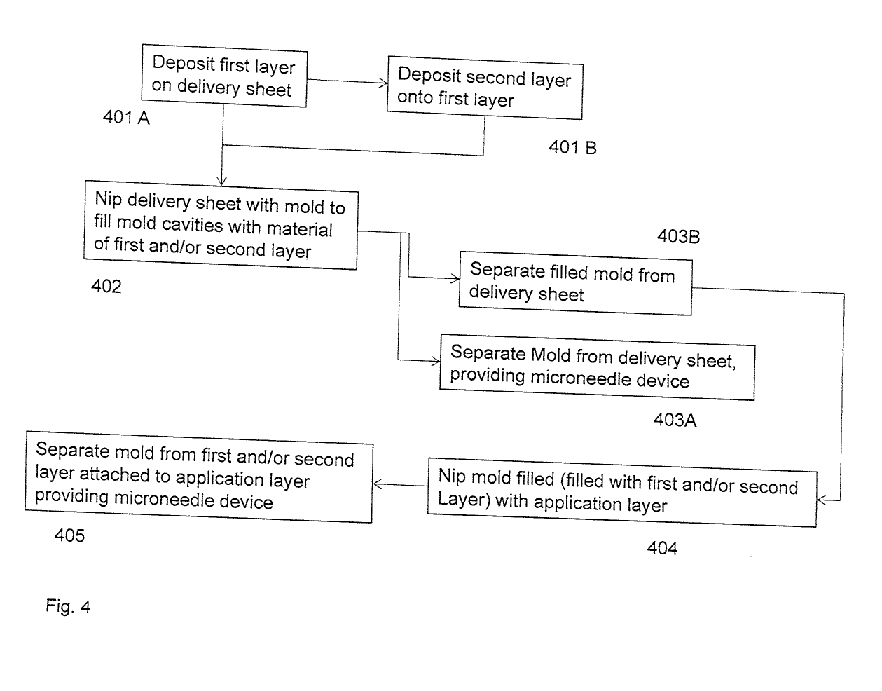

As shown in FIG. 4, the present invention also includes a system for fabricating microneedle device 300. The continuous system can include a thin-film roll-to-roll system or a sheet based system. As shown in FIG. 4, the first step 401 includes depositing the materials to become the microneedles onto the delivery sheet. In an alternative embodiment, 401B, a second layer of a second material can be deposited onto the first layer. Next, the deposited material on the delivery sheet is mated with a mold in a nip-point 402. Next, at step 403A, the mold is removed from the material laminated to the mold, thereby revealing microneedle device with needles that mimic the shape, size, and three-dimensional profile of the cavities of the mold. In an alternative embodiment, step 403B, the mold/material of the first and/or second layer combination is separated from the delivery sheet. Next, step 404, the mold/material of the first and/or second layer combination is run through a nip-point with application layer to adhere the first and/or second layer with the application layer. The nip-point for mating first and/or second layer with application layer can be either (i) a second pass through the first nip-point or (ii) a second nip-point configured in series with the first nip-point. Finally, at step 405, the first and/or second layer mated with the application layer is removed from the mold, thereby providing microneedle device with needles that correspond in size, shape, and profile to the cavities in the mold and correspond in composition to the first and/or second layers.

An important aspect of the present invention is the advantages of the present invention roller based fabrication system for manufacturing microneedle devices over the prior art. The present invention roller based manufacturing system of the present invention provides highly uniform laminate layers, for example, layer thickness uniformity disclosed herein, which provide highly uniform and controllable drug loading of the microneedles. Another advantage of the roller based fabrication system of the present invention is the fabrication rate of microneedles where the system can generate, depending on the line speed achievable of the roll-to-roll system. In a system with line speed limited to 0.1 ft/min and at a line width of 1 inch, the system would generate 0.5 square feet of microneedles per hour. In a system with line speed approaching 150 ft/min and at a line width of 72 inches, the system would generate 54000 square feet of microneedles per hour. In a preferred embodiment of the present invention, the roll-to-roll system will run at a line speed of between 1 and 50 ft per minute with a line width between 6 inches and 24 inches, thereby producing between 30 and 6000 square feet of microneedles per hour. Table 1, below, shows exemplary calculations of square feet of microneedles prepared per hour based on varying line speeds and associated web widths.

TABLE 1
Square Feet per hour of microneedles produced.
Sq Ft per hour at various line speeds and web widths

| Line Speed | Web Width (in) | | | | |
|---|---|---|---|---|---|
| (ft/min) | | 6 | 12 | 24 | 72 |
| 0.1 | .5 | 3 | 6 | 12 | 36 |
| 1 | | 30 | 60 | 120 | 360 |
| 10 | 0 | 300 | 600 | 1200 | 3600 |
| 50 | 50 | 1500 | 3000 | 6000 | 18000 |
| 100 | 00 | 3000 | 6000 | 12000 | 36000 |
| 150 | 50 | 4500 | 9000 | 18000 | 54000 |

Also disclosed herein is the number of patches formable per hour based on a given line speed and associated web width, for a patch size of 1 square inch, as shown in Table 2. Accordingly, for a line speed of between 1 and 50 feet per minute from a web width of between 6 and 24 inches, the number of 1 square inch patches produced per hour would be between 3600 and 72000.

TABLE 2
Exemplary number of patches produced per hour given a
1 square inch patch size.
Number of patches per hour at various line speeds and
web widths for a specified patch size

| PATCH SIZE | NUMBER OF PATCHES/HR AT | | | | |
|---|---|---|---|---|---|
| (sq in) 1 | SPECIFIED PATCH SIZE | | | | |
| Line Speed | Web Width (in) | | | | |
| (ft/min) | | 6 | 12 | 24 | 72 |
| 0.1 | | 36 | 72 | 144 | 432 |
| 1 | 0 | 360 | 720 | 1440 | 4320 |
| 10 | 00 | 3600 | 7200 | 14400 | 43200 |
| 50 | 000 | 18000 | 36000 | 72000 | 216000 |
| 100 | 000 | 36000 | 72000 | 144000 | 432000 |
| 150 | 000 | 54000 | 108000 | 216000 | 648000 |

The present invention discloses controlled deposition of the first and/or second material (100,200) on the delivery sheet 105. Controlled deposition means depositing the first and/or second material with precision control over uniform homogeneous composition mixture and precision control the thickness and defect free conditions. An important aspect of the present invention is uniform composition, drug (active agent) loading, size, mechanical properties, size, and shape of each needle in the resulting microneedle device that includes tens, hundreds, and/or thousands or more of needles per device. The first and/or second material, according to the present invention, are deposited onto delivery sheet with nanometer precision in deposition thickness and/or uniformity across the entire land-area, where such land-area may be up-to or greater-than three feet wide and continuous for greater than one hundred (100) feet in length. In another embodiment, the uniformity of thickness of first and/or second material is within 0.1 percent of the height of the microneedles (depth of the mold cavity). In another embodiment, the uniformity of thickness of first and/or second material is within 0.5 percent of the height of the microneedles (depth of the mold cavity). In another embodiment, the uniformity of thickness of first and/or second material is within 1 percent of the height of the microneedles (depth of the mold cavity). In another embodiment, the uniformity of thickness of first and/or second material is within 2 percent of the height of the microneedles (depth of the mold cavity). In another embodiment, the uniformity of thickness of first and/or second material is within 5 percent of the height of the microneedles (depth of the mold cavity). In another embodiment, the uniformity of thickness of first and/or second material is within 10 percent of the height of the microneedles (depth of the mold cavity). It is important to note that the uniformity and consistency between each needle of a microneedle device in physical size (such as, for example, width, 3-dimensional shape, height, tip shape and/or dimension, and the like), chemical and/or mechanical properties and the like is important for at least the reasons of providing a drug delivery device with uniform drug loading and uniform tissue penetration, both of which are ultimately important for uniform drug delivery to a patient. Many prior art devices and manufacturing methods and systems fail to bring the consistency and uniformity to each respective needle of microneedle drug delivery devices for predetermined, controlled and uniform delivery of a desired agent as do the methods, systems and devices of the present invention.

Another important aspect of the present invention is the tip of each needle of the microneedle device. The tip region of each needle is responsible for piercing the tissue through which the device is intended to deliver the active component or attract or collect an unwanted component. According to the prior art, general tip diameters range in the 10 micrometer or larger range. According to some embodiments of the present invention, tip of each needle of microneedle device is formed from the deepest region 127 of the cavity 125 in mold 120 and is formed by preparing the mold from a master template. Therefore, a part of the present invention include developing master templates with shape and size micro and nano structures that will result in needles with the desired shape and size. Accordingly, the tip diameters of the needles of the present invention microneedles can be less than 10 micrometers in diameter. In another embodiment tip of microneedle formed from the deepest region of the cavity 127 in mold cavity 125 can be less than about 5 micrometers in diameter. In another embodiment, tip of microneedle formed from the deepest region of the cavity 127 in mold cavity 125 can be less than about 1 micrometer in diameter. In another embodiment, tip of microneedle formed from the deepest region of the cavity 127 in mold cavity 125 can be less than about 500 nanometers in diameter. In another embodiment, tip of microneedle formed from the deepest region of the cavity 127 in mold cavity 125 can be less than about 250 nanometers in diameter. In some embodiments, each needle of microneedle device of the present invention is formed from an independent needle or isolated particle on a film and each needle is formed from a particle designed with micrometer and/or nanometer precision. According to such embodiments, each needle can be an isolated particle positioned on a film where each particle has a tip width of less than 100 nanometers. In other such embodiments, each needle can be an isolated particle positioned on a film where each particle has a tip width of less than 250 nanometers. In other such embodiments, each needle can be an isolated particle positioned on a film where each particle has a tip width of less than 500 nanometers. In other such embodiments, each needle can be an isolated particle positioned on a film where each particle has a tip width of less than 750 nanometers. In other such embodiments, each needle can be an isolated particle positioned on a film where each particle has a tip width of less than 1 micrometer. In other such embodiments, each needle can be an isolated particle positioned on a film where each particle has a tip width of less than 5 micrometers. In other such embodiments, each needle can be an isolated particle positioned on a film where each particle has a tip width of less than 7.5 micrometers. In a preferred embodiment, the tip diameter of the microneedle is selected based on input factors including, but not limited to, microneedle modulus, microneedle material, active agent contained in or on microneedle, application site, etc to obtain the desired delivery.

### PRINT Microneedles

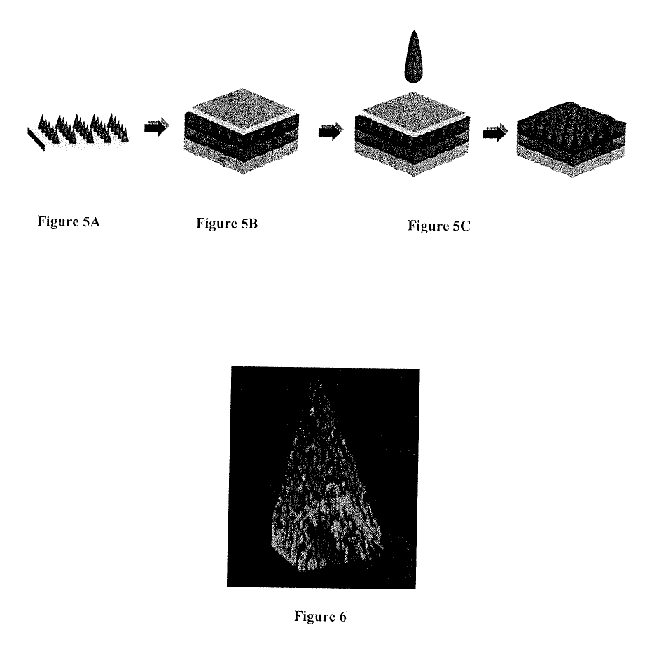

An approach to microneedle fabrication using a high throughput roll-to-roll process is disclosed. An array of distinctive, individual microneedles is manufactured and collected on a dissolvable substrate, such as for example a water-soluble substrate (FIG. 5A). In certain embodiments, the substrate can be flexible, allowing for the microneedle devices to be “rolled” into the skin, whereas the needles themselves are stiff. Microneedles of the present invention can have a modulus between 1-10 GPa to provide adequate stiffness. The flexibility of the substrate of the present invention allows the array of highly-dense, stiff microprojections to overcome the “bed of nails” effect and pierce the elastic epidermis more efficiently than rigid arrays of microneedles. Substrates could have a modulus of 0.1-1 GPa; systems were the difference in modulus of the microneedle material and the substrate is greater than a factor of 2 or more, preferably 10 or more. After application to the skin, the needle patch would remain in the skin to allow the needles to dissolve (FIG. 5B). In one embodiment, the substrate would then be dissolved with the appropriate solvent, such as for example through use of a damp cloth, leaving the entire microneedle array in the skin (FIG. 5C). In this configuration, the entire payload of drug in the microneedle patch would be delivered to the skin. By creating microneedle molds compatible with the PRINT process, arrays of soluble, highly-reproducible, stiff, highly-uniform, large-area microneedles can be made and collected onto a soluble harvesting sheet. The system for molding the microneedles in molds of the present invention does not require vacuum or centrifugation steps such as the systems of the prior art.

The microneedle arrays of the present invention are fabricated in rapid processing. According to an embodiment of the present invention the microneedle array can be fabricated in less than 10 minutes. According to an embodiment of the present invention the microneedle array can be fabricated in less than 5 minutes. According to an embodiment of the present invention the microneedle array can be fabricated in less than 2 minutes. According to an embodiment of the present invention the microneedle array can be fabricated in less than 1 minute. According to an embodiment of the present invention the microneedle array can be fabricated in less than 30 seconds. PRINT microneedle fabrication of the present invention can be adapted on any scale of production; this particular advantage allows for patches of virtually any size to be made affordably and quickly.

The microneedle devices of the present invention can be applied transdermally to treat a wide variety of conditions, including but not limited to, breast cancers, skin cancers, vaccines, chronic skin conditions, routine injections, anti-inflammatory delivery, wound healing, or cosmetic applications. The microneedles for such applications can be made of a variety of compositions, including but not limited to polymers, monomers, sugars, drugs, small molecules, nucleic acids, or any additional agent (i.e. active agent) of interest.

Microneedles made from the aforementioned materials loaded with micro- and nanoparticles are one embodiment of the present invention. In an embodiment, the particles can also be made by the PRINT process described herein. The micro and/or nanoparticles used to fill the cavities and finally become the needles of the microneedle device of the present invention can be made of a wide range of chemical compositions, including but not limited to polymers, monomers, sugars, drugs, small molecules, nucleic acids, or any additional agent (i.e. active agent) of interest. Microparticles, 1 μm cylinders, made of bovine serum albumin have previously been loaded into microneedles according to an embodiment of the present invention (see FIG. 6). Microneedles such as these can be used as “mother-ship” delivery vehicles to release their cargo into the skin for local or systemic delivery of the particles. The needle matrix will determine release rate of the particle into the skin. The particle can be used for a wide variety of purposes, including but not limited to, releasing drug, releasing a biologic agent, scavenging, delivering Red Blood Cell (RBC) mimic particles, etc. In the embodiment that the particles are delivering a therapeutic, the particle matrix will determine its release rate in vivo.

In another embodiment, stratified microneedles can be made. In such needles, the tip will preferentially be loaded with a matrix that contains the desired cargo. Next, a plug of innocuous material can comprise the base of the needle. The plug can have multiple functions, including but not limited to, enabling the selective delivery of the cargo to a more narrow deposition depth, enabling accurate and reliable loading of the cargo, enabling the “hole” left in the skin to be filled with a protective agent to prevent bacteria from invading, etc. The composition of the tip, the plug, and the cargo can be a wide range of materials, including but not limited to polymers, monomers, sugars, drugs, small molecules, nucleic acids, or any additional agent (i.e. active agent) of interest.

An example of an attractive breast cancer target for an embodiment of the present invention is inflammatory breast cancer (IBC). IBC is the most aggressive form of invasive breast cancer known. Unlike many breast cancers that present as a lump, IBC dysplastic cells commonly reside in the dermal lymphatics, causing obstruction to lymphatic drainage and “inflamed” skin. Much research on IBC treatment has focused on improving systemic therapies. In spite of these efforts, clinicians have recognized the complexity of IBC and have stated that prognosis of these patients remains poor. As innovative strategies are critical, a novel transdermal-based approach could serve as an avenue for a local and possibly systemic, yet minimally invasive, therapy. PRINT microneedles loaded with pertinent therapeutics could offer an attractive solution to improve the efficacy of existing IBC therapies while reducing the deleterious effects commonly associated with traditional injections. Skin cancers such as lentigo maligna melanoma or superficial basal cell carcinoma, types of in situ melanoma and carcinoma associated with prolonged sun exposure, could be attractive targets for the microneedle devices of the present invention. Both cancers are routinely located on the face and other sensitive areas, have ill-defined clinical margins, and surgical excisions are often associated with a high level of risk. Transdermal treatments may be enhanced by the use of an embodiment of microneedle devices of the present invention with large surface areas and adequate flexibility. Possible therapeutics that could be adapted to the present invention to treat these cancer include, but are not limited to, small molecule chemotherapeutics (i.e. docetaxel, paclitaxel, cisplatin, carboplatin, doxorubicin, daunorubicin, epirubicin, capecitabine, gemcitabine, fluorouracil, imiquimod, vismodegib, etc.) and biologic agents (i.e. monoclonal antibodies (trastuzumab, bevacizumab, lapatinib, ipilimumab, etc.), and fragments thereof, interferon, interleukin-2, siRNA, etc.).

The microneedle devices of the present invention can be useful for the administration of vaccines, for many vaccines are large, fragile biologics that could be incorporated into microneedle matrices for successful delivery through the skin. Microneedles for the treatment of conditions that require frequent injections, such as the administration of human growth hormone or insulin, are also attractive avenues for the application of the present invention. These injections are commonly associated with pain and low patient compliance which may be overcome by the use of an embodiment of the present invention. Vaccines against diseases including but not limited to influenza, dengue, malaria, hepatitis, measles, mumps, rubella, diphtheria, tetanus, polio, varicella, HIV, HPV, and cancers, etc., are an embodiment of the present invention. Vaccination strategies that may be adapted to the present invention for the vaccination of diseases include, but are not limited to, whole attenuated pathogens, subunit vaccines, conjugates, recombinant vaccines, and the delivery of RNA replicon, antigens, and adjuvants, etc.

Microneedle devices of the present invention may aid in the delivery of local anti-inflammatory medications or treatments for chronic skin conditions and autoimmune disorders, such as psoriasis, rosacea, pemphigus, keloids, rheumatoid arthritis, etc. Specific treatments include corticosteroids, dexamethasone, or monoclonal antibodies (i. e. Humera, etc.). Certain medications traditionally delivered via a subcutaneous route, including heparin, a blood anticoagulant, lidocaine, a local anesthetic, or epinephrine, a treatment for anaphylaxis, may show efficacy when delivered transdermally via an embodiment of the present invention. Microneedle patches of the present invention may increase the efficacy of medications usually delivered in the form of traditional flat transdermal patches or creams by increasing the permeability of the skin, such as nicotine. PRINT microneedles may be of use in wound healing applications, for the scavenging of surface-deep biomolecules at the site of the wound may be advantageous. Specifically, microneedles that have an affinity for pro-inflammatory molecules like cytokines and chemokines would be advantageous. Treatments used for cosmetic applications, like Botox and hyaluronic acid, often used on the face, may be delivered in an active form by microneedle devices of the present invention. Due to the tunable size of the microneedle patches of the present invention, the large surface area aforementioned applications would be able to be fully treated with PRINT microneedles.

According to embodiments of the present invention, a process for making silicon master templates via tilted, rotated photolithography is disclosed herein. The mask dimensions as well as the incident angle of the light determined the dimensions of the structures which will ultimately be replicated into microneedles through the molding process as described herein. A positive replica of the master template is then made as an intermediate. The positive replicas are made using PDMS due to its low surface energy, ease of use, high flexibility, and low cost. The positive replicas made from PDMS have dimensions that mirror the cavities of the master templates which ultimately become the microneedles. The positive replicas are then used to make PRINT-compatible molds from a photocurable perfluoropolyether (PFPE) elastomer.

Microneedles are then fabricated using the adapted PRINT process of the present invention. According to one embodiment of the present invention, first, polyvinylpyrrolidone (PVP) is selected as the polymeric matrix and cast as a film onto a sheet of plastic. The complex is then passed through a heated laminator. After separation, individual PVP microneedles remained in the mold. Next, according to an embodiment of the present invention, the microneedles are collected onto a water-soluble, flexible harvesting film. Microneedle arrays of reproducible needles were fabricated, maintaining the geometry of the mold and original master template. In particular, the tip radius of curvature of the needles remained under 10 μm. The arrays are flexible and easily removable from the mechanical layer necessary for production. Therefore, completely dissolvable microneedle patches have been successfully fabricated.

To show the efficacy of the microneedle patches of the present invention to penetrate the epidermis for therapeutic delivery, the devices were tested on ex vivo mouse and human skin. Flexible patches were “rolled” on and pressed into the skin with the gentle force of a thumb. Three different experimental conditions were compared: control (no microneedles applied), patches left in the skin for 10 seconds and then removed, and patches left in the skin for 10 minutes followed by the dissolution of the substrate with water. Microneedle patches applied to the skin for 10 seconds and then removed showed that epidermal penetration was achieved with this method. The skin was visually assessed to observe epidermal breach via light microscopy; holes in the pattern of the patch can be seen in the skin. Microneedle patches applied for 10 minutes showed the 100% dissolvable character of this embodiment of the present invention as well as epidermal penetration and the successful delivery of a drug surrogate.

### Example of Microneedle Fabrication According to an Embodiment of the Present Invention

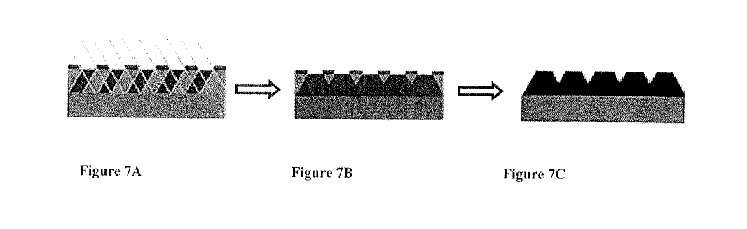

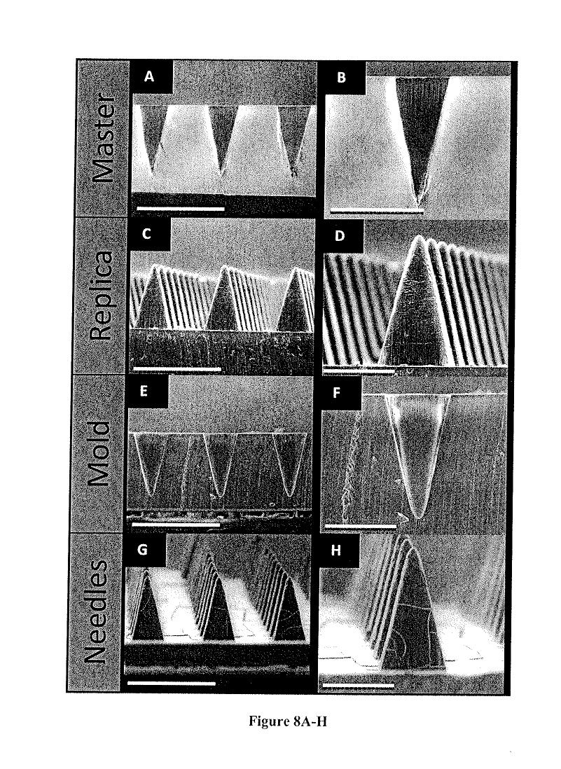

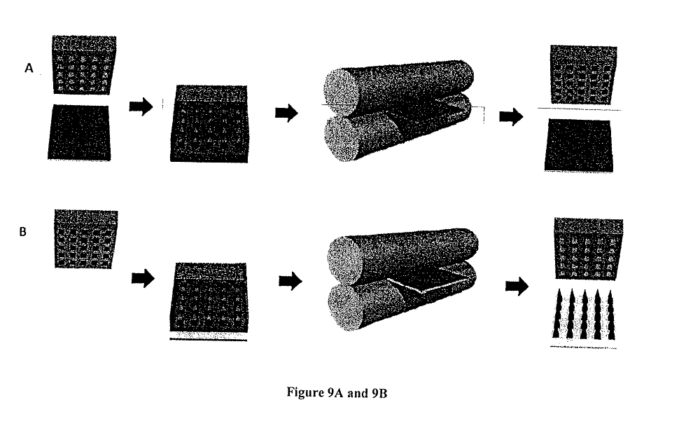

To manufacture PRINT microneedle patches, master templates were first prepared using a tilted-rotated photolithography approach adapted from Han et al. Rigid SU-8 2150 microneedle templates were fabricated using a tilted-rotated UV lithography approach. In summary, a single crystalline Si wafer was coated with an antireflective coating consisting of a CrOₓ/Cr multilayer. The thickness of the CrOₓ layer was chosen to minimize reflections of 365 nm UV light from the substrate. The substrate was then spin-coated with 600 μm thick SU-8 and soft baked at 100° C. for 8 h. The coated Si wafer was cleaved into squares pieces, which were then attached to a light-field mask of 200 μm×200 μm chromium squares and 200 μm spacing. The substrate was then exposed to filtered UV light incident at angles between 18-25° (FIG. 7A-7C). Both the mask dimensions and the incident angle of UV light determine the depth of the mold, and ultimately, the length of the microneedles. The exposure was performed in four 450 mJ/cm² increments in which the substrate was rotated 90° about its surface normal between each exposure. The post-exposure bake (PEB) was performed at 65° C. for 30 min. At the end of the PEB, the temperature was slowly ramped down to room temperature and the substrate was allowed to relax for 60 min. The unusually low-temperature PEB and the subsequent gentle cooling down steps were critical to reduce stress in the SU-8, which can cause the mold to break. The substrate was then developed with propylene glycol monomethyl ether acetate (PGMEA) in an ultra-sonic bath for 10 min and rinsed with isopropanol. This development sequence was repeated three times to ensure the molds were fully developed. According to further embodiments, master templates can be fabricated from other known techniques in the art, such as for example, photolithography, soft lithography, light etching or photo curing of material, electron beam etching or curing of material, additive manufacturing, stereolithography or the like, such as techniques disclosed in US patent application number 20130295212 to Y. Chen and C. Zhou; and “Development of a Multi-material Mask-Image-Projection-based Stereolithography for the Fabrication of Digital Materials” to C. Zhou, Y. Chen, Z. Yang, and B. Khoshnevis, available at http://utwired.engr.utexas.edu/lff/symposium/proceedingsArchive/pubs/Manuscripts/2011/2011-06-Zhou.pdf, the contents of each are herein incorporated by reference in their entirety.

These templates were imaged via Environmental Scanning Electron Microscopy (ESEM) to determine the length and tip radii of curvature that would be achieved through replication. Seen in FIG. 8A-8B, the template used for this study was 360 μm in length and had tip radii of curvature under 10 μm. This length was selected based on the desire to reach the viable epidermis after piercing the stratum corneum.

A positive replica of the master template was made as an intermediate. The replicas were fabricated using commercially available polydimethylsiloxane (PDMS) due to its low surface energy, ease of use, high flexibility, and low cost. A thick layer of silicone (Sylgard 184, Dow Corning) was cast over the master. The PDMS was degassed in a vacuum desiccator for 2 h before centrifugation for 20 min at 3000 g and 4° C.; this process was then repeated once. The replica was left to cure under vacuum overnight at room temperature (RT) and was finished with a 2h bake in a 65° C. oven. The replicas showed notable reproducibility of the master templates, having comparable needle lengths and tip radii of curvature via ESEM (FIG. 8C-8D).

The positive replica was then used to make PRINT-compatible molds from a photocurable perfluoropolyether-dimethacrylate (PFPE-dMA) elastomer with a molecular weight of 4 kDa. PFPE is non-wetting and non-swelling, resulting in molds with a highly fluorinated surface that allow for microneedles of diverse chemical compositions to be made. A 0.2 wt % solution of 2,2-diethoxyacetophenone in PFPE-dMA was drop cast onto the replica, and a flexible plastic sheet was applied to serve as a supportive backing. The mold was cured in nitrogen-purged UV oven (λ=365 nm), and the finished mold was separated from the replica for use. The PRINT molds are consistent with the dimensions of the replicas, reproducibly mimicking the SU-8 master templates (seen via ESEM, FIG. 8E-8F). It should be noted that, based on laboratory findings, each master template can be used to make hundreds of PDMS replicas, and each replica can be used to make at least 50 PFPE molds. Each PFPE mold can be used to create at least 10 microneedle arrays via PRINT processing.

The substrate for the microneedle backing was designed to be flexible and water-soluble. This is desirable for two reasons: 1) to facilitate improved penetration of the stratum corneum by avoiding the “bed of nails” effect, and 2) to create a microneedle patch that is 100% dissolvable to eliminate sharp, hazardous biowaste. A matrix of Luvitec VA64, a polyvinylpyrrolidone/polyvinylacetate blend, was selected due to its high water solubility and biocompatibility for topical use. Thick films of this polymer cast in methanol were not sufficiently flexible; therefore, multiple plasticizers were studied to lower the glass transition temperature (T_{g}) of the film to impart flexibility. Plasticizers studied included glycerol, castor oil, Tween80, PEG400, triethyl citrate, tributyl citrate, and trimethyl citrate at loadings of 1-10 wt %. These small molecules were mixed in methanol at 30 wt % loadings, cast upon plastic sheets, and allowed to dry for 24 h at RT. In particular, triethyl citrate and trimethyl citrate in 1-3 wt % loadings showed promise for use as substrates.

These films were analyzed by thermal gravimetric analysis (TGA) and differential scanning calorimetry (DSC). TGA studies were done to determine the 95% degradation temperature of the materials to avoid decomposition in the DSC. TGA decomposition experiments were done by heating 5-10 mg of substrate from 0-550° C. at 10° C./min, and the 95% decomposition temperature was determined; the upper temperature limit for the DSC experiments was to be no more than 50° C. lower than the 95% decomposition temperature for each material. DSC was used to determine the T_{g}'s of the substrates. Samples (5-10 mg) were crimped into aluminum pans and heated from −20° C. to 100-120° C. at a rate of 5° C./min, cooled at a rate of 10° C./min to −20° C., and heated again in a second cycle. T_{g}'s were determined from the second heating cycle. A glass transition temperature around 25° C. was seen for the triethyl citrate films with loadings of 1-3%; this T_{g} allowed for optimal flexibility and thermal stability at room temperature. Therefore, the blend of Luvitec VA64 in methanol and 2 wt % loading of triethyl citrate was selected for the fabrication of optimal substrates. Select substrates were loaded with 0.5 wt % fluorescein dye for imaging purposes.

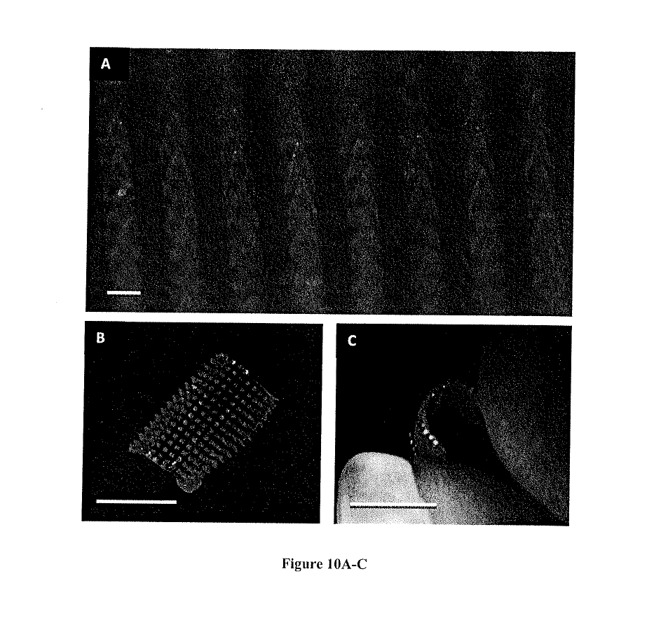

Polymeric microneedles were then fabricated using the PRINT process (schematic shown in FIG. 9A-9B). While the present invention can be applied to fabricate microneedles out of a wide variety of chemical compositions, polyvinylpyrrolidone (PVP) was selected as the first matrix for study. This polymer was chosen because it is highly water soluble, has a high tensile strength, and is a biocompatible, FDA approved pharmaceutical excipient. PVP with a molecular weight of 10 kDa was used because it has been shown that masses less than 20 kDa are cleared efficiently from the kidney after subcutaneous injection and, therefore, are safe for human use. Rhodamine B dye at a loading of 0.1% was included in the matrix as a drug surrogate by mixing it into the PVP/water solution before film casting. A film (˜380 μm thick) was mated to the PFPE mold, covered with a plastic sheet, and passed through a heated nip at 105° C., filling the mold. The complex was cooled to RT and the plastic sheet was removed. Due to the non-wetting characteristic of the PFPE molds, excess PVP was wicked away leaving arrays of discrete microneedles. The filled mold was mated to the aforementioned flexible, water-soluble substrate, covered with a plastic sheet, and passed through a heated nip at 65° C. The mold and plastic sheet were then removed, leaving a 100% water soluble microneedle patch.

While heated fabrication was used to make the present invention, PRINT is also compatible with photocurable systems, allowing for room temperature fabrication when needed for thermally-labile cargos. For these studies, fabricated patches contained approximately 700 needles; however, the PRINT process is highly scalable for cost-effective manufacturing, enabling patches of virtually any size to be created affordably and quickly.

The microneedles were characterized by ESEM (FIG. 8G-8H) and macroscopic brightfield imaging (FIG. 10A-10C). Microneedles demonstrated remarkable reproducibility (FIG. 10A), with bases measuring 195.1±4.4 μm, lengths of 361.4±5.7 μm, and tip radii of curvature of 9.93±1.7 μm (n=15). These dimensions also closely mimic the master template, indicating that the microneedles retained their original shape and sharpness throughout processing. The flexibility of the array can be seen in FIG. 10B-10C. The rigid microneedles remained intact after the gentle bending of the array by hand. Both the microneedles and the substrate were seen to dissolve rapidly in the presence of a few drops of water; after 5 min, the device was completely dissolved. Therefore, novel 100% water-soluble microneedle patches on flexible substrates can be made quickly and reproducibly via PRINT processing.

The present invention microneedle arrays were tested in ex vivo murine skin samples (UNC Animal Core Facility). All skin samples were received and stored at −80° C. until testing occurred. Prior to experimental studies, the skin samples (in Eppendorf tubes) were thawed briefly in 37° C. tap water. The thawed samples were then pinned over corkboard and blotted dry to simulate in situ conditions. Flexible patches were “rolled” on and pressed into the skin with the gentle force of a thumb and then rolled with a hand roller. Three different experimental conditions were compared: control (no microneedles applied), patches left in the skin for 10 s and then removed, and patches left in the skin for 10 min followed by the dissolution of the substrate with water.

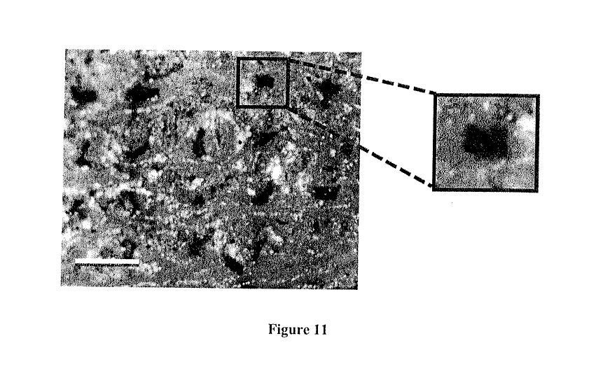

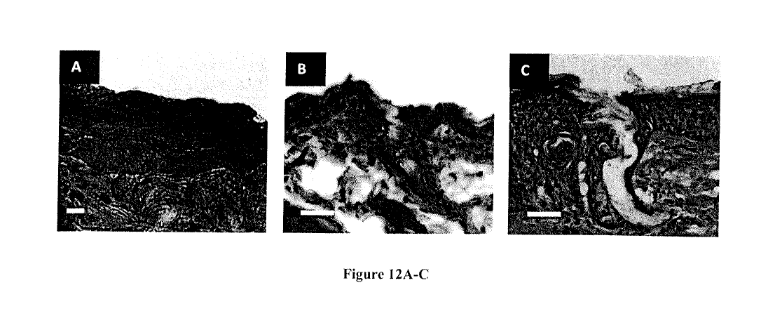

Initial testing assessed the ability of the microneedles to successfully penetrate the stratum corneum of the murine skin samples. For this evaluation, all patches left were in the skin for 10 s, removed, and a green tissue marking dye was immediately applied to the skin for 5 min and subsequently wiped off so that locations of skin penetration could be identified macroscopically. Commercially available green tissue-marking dye (Cancer Diagnostics) was prepared by diluting the solution in a 1:1 mixture with isopropanol. FIG. 11 shows a greyscale image of a murine skin specimen after the application of microneedles for 10 s. The locations of epidermal breach can be seen on the skin; this was verified by histology. Additionally, the microneedles showed evidence of dissolution within the skin after 10 s. The drug surrogate could be visually perceived within sites of microneedle insertion and could not be wiped from the surface. Further brightfield macroscopic images of the patches after removal also showed at least half of the microneedle length had dissolved within this 10 s time.

After verifying that the microneedles could pierce the stratum corneum, further studies were conducted to evaluate the complete dissolution of the microneedle patches of the present invention and release of the drug surrogate. For these studies, all patches were left in the skin for 10 min. The patch was rolled for 1 min and then left for 9 min at ambient conditions. The patch backing was then dissolved with a small aliquot (<200 μL) of tap water. Within 5 min, the entire substrate (loaded with fluorescein) was dissolved and the skin was wiped clean. No further dyes were applied. Rhodamine B was easily visible within the skin; the dye was not localized to the site of microneedle insertion but, rather, was present throughout the skin, suggesting that the drug surrogate was able to diffuse within the skin after 10 min.

The skin samples from all aforementioned experiments (including controls) were then fixed for 2 h in 2% paraformaldehyde (PFA) and left overnight in 15% sucrose in 1×PBS at 4° C. PFA was prepared by diluting a commercially available solution of 4% PFA (USB) in PBS with additional 1×PBS (Sigma) in a 1:1 mixture. Tested skin samples were then embedded in Tissue-Tec® Optimum Cutting Temperature medium and cryosectioned. Sections (12 μm) were taken at −25° C. based on manufacturer suggestions. Half of the sections were set aside for imaging using fluorescent microscopy; these sections were fixed briefly for 10 s in FROZEN-FIX prior to coverslipping. The remaining sections were hematoxalin and eosin (H&E) stained for brightfield microscopy imaging. Staining was done using the procedure outlined by Cancer Diagnostics for the CRYO-KIT prior to coverslipping.

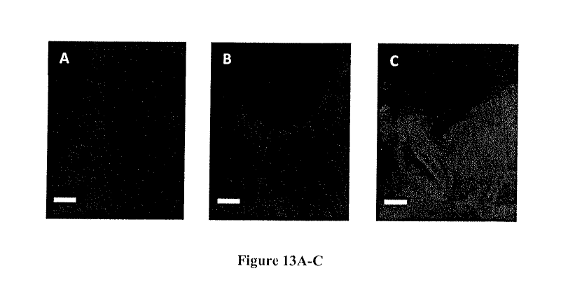

After H&E staining and brightfield imaging, the control samples did not show any epidermal breach as expected (FIG. 12A); the skin was consistently smooth. Evidence of epidermal breach was seen in skin sections from both the 10 s and 10 min experiments, shown in FIG. 12B-12C by the breaking of the stratum corneum (the outer epidermal layer seen as dark purple). The penetration depths of the microneedles observed were consistently shorter than the lengths of the microneedles, but the insertion depth was longer for the 10 min tests. This is believed to be due to the elasticity of the skin and geometry of the needles themselves. However, it is promising that the depth of the needle penetration seen increased when the patches were applied for 10 min, which more accurately reflects the ultimate intended clinical application of the 100% dissolvable patch.

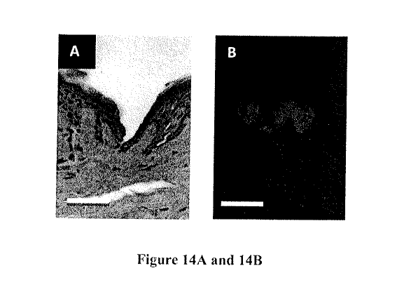

Images of the unstained skin via fluorescent microscopy showed the efficiency of the drug surrogate delivery to the skin. Seen in FIG. 13, a large qualitative difference in fluorescence intensity was observed among the three samples. While the control showed no fluorescence (FIG. 13A), an observable fluorescence was seen in the 10 s test in selective areas of the skin (FIG. 13B). Comparatively, considerably higher fluorescence intensity within the skin was seen for the 10 min time period throughout the whole skin section (FIG. 13C). This confirms that the drug surrogate was released from the needles and diffused beneath the stratum corneum throughout the duration of the patch application.

In addition to optimization and validation in murine skin samples, pilot studies to determine the ability of the PRINT microneedles to pierce human skin were also conducted. Human tissue excised from a patient with inflammatory breast cancer (IBC) was obtained from the Cooperative Human Tissue Network (CHTN). The conditions and procedures used for the murine tests were replicated on these tissue samples. Preliminary results indicate that epidermal breach and subsequent drug surrogate release are also seen when done on human skin specimens. FIG. 14 shows a site of microneedle penetration and corresponding rhodamine fluorescence in IBC skin. As compared to the results obtained with the murine model, these results suggest that the drug surrogate release kinetics are slower in human skin than in murine skin, which was anticipated due to the increased thickness of human skin. While further optimization of the procedure and conditions will need to be done prior to clinical translation, these findings support the proof of concept that microneedles of the present invention may be used to penetrate human skin and deliver loaded cargo.

### Example

According to certain embodiments of the present invention, the PRINT microneedles of the present invention can scavenge for biomolecules in the epidermis, in particularly embodiment the biomolecules can be nucleic acids as described. The microneedles are cationic, or imparted with a positive charge, so they will be able to attract negatively charged biomolecules—specifically, for example, the negatively charged phosphate backbone of nucleic acids. Furthermore, the microneedle matrices are porous to absorb and retain scavenged material. The cationic scavenging microneedles provide a microneedle that can be clinically applied to scar and burn healing and even cancer screening and the like.

According to particular embodiments, a proper blend of polymers and additives composing the microneedle composition that expresses the properties necessary for scavenging is disclosed. The thermodynamic properties of the polymer blend used to fabricate the microneedles were analyzed computationally and via thermogravimetric analysis (TGA) and differential scanning calorimetry (DSC) to assess its strength for skin piercing. Next, the fabrication process for the selected chemical formulation was optimized and selected patches were imaged using environmental scanning election microscopy (ESEM) to observe needle sharpness and the reproducibility of the microneedle patch. Lastly, after the fabrication process was optimized, the microneedle patches were analyzed for their ability to pierce skin. The microneedles may also attract cy-5 labeled DNA in situ and/or in vivo.

Microneedle Polymer Blend Matrix

TABLE 1
Summary of constituents of polymeric matrix
for cationic scavenging matrices.

| Constituent | Weight Percent |
|---|---|
| Polyethylene glycol | 77% |
| diacrylate/dimethacrylate | |
| 2-(dimethylamino)ethyl | 20% |
| methacrylate | |
| Fluoresecein o-acrylate | 2% |
| 2,2-diethoxyacetophenone | 1% |

In particular embodiments of the present invention, the composite matrices of the microneedles include, but are not limited to, a blend of chemicals comprised mainly of polyethylene glycol.

In embodiments, the polyethylene glycol derivative component is from about 51% to about 99%; from about 60% to about 90%; from about 70% to about 80% by weight percent. The amount of so-called chemical handle described below that imparts a positive charge can be added in relative proportions to the PEG component. In embodiments, this compound is present from about 0.9:1 to about 1:10 relative to the PEG component. In embodiments, this compound is present from about 0.7:1 to about 1:7 relative to the PEG component. In embodiments, this compound is present from about 0.5:1 to about 1:5 relative to the PEG component. In embodiments, this compound is present from about 0.4:1 to about 1:4 relative to the PEG component. This compound is preferably (dimethylamino)ethyl methacrylate (DMAEM).

As used herein, short crosslinkers refer to derivatives of PEG400-600, such as PEG₅₇₅diacrylate and PEG₅₅₀dimethacrylate. As used herein, long crosslinkers refer to derivatives of PEG above 600, such as PEG650-1000, or PEG 700-800. Examples include PEG₇₀₀diacrylate and PEG₇₅₀dimethacrylate.

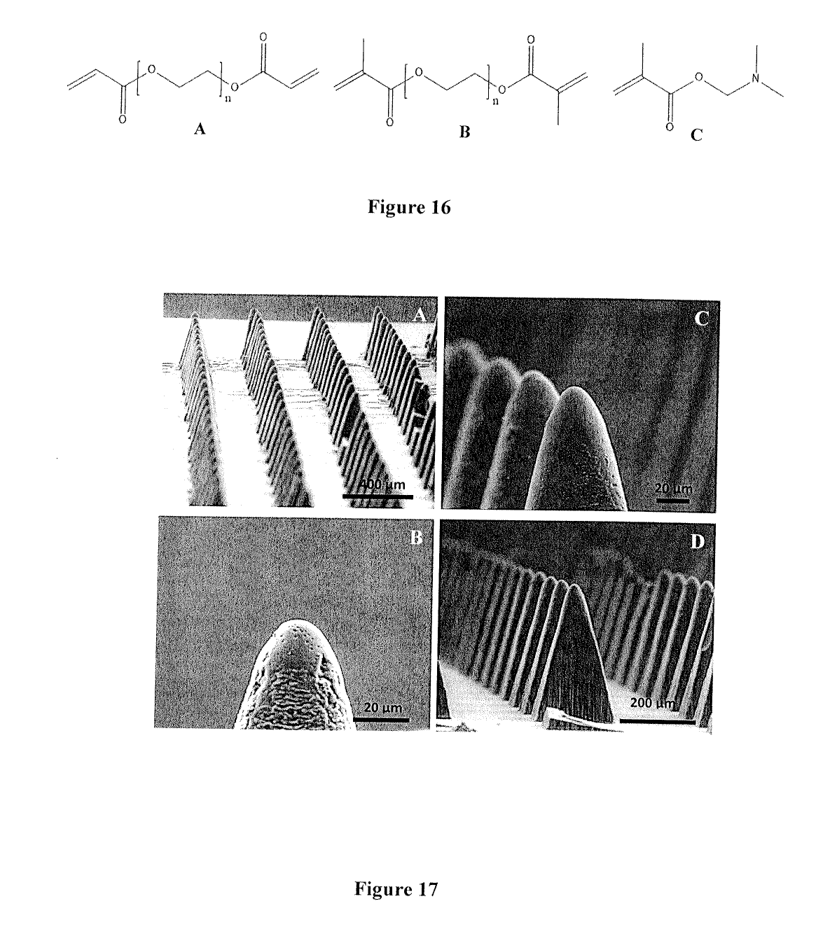

FIG. 16 shows chemical matrix compositions and Table 1 summarizes the constituents of the blend used for the microneedle matrix, according to an embodiment of the present invention. Polyethylene glycol diacrylate (PEG diacrylate) and dimethacrylate (PEG dimethacrylate) are oligomers of ethylene oxide that have been end terminated on both ends with acrylate or methacrylate groups, respectively. When bulk polymerized, PEG diacrylate and PEG dimethacrylate form a crosslinked microneedle matrix, or a mesh-like structure, pores are created within the microneedle matrix material to store scavenged extracted biomolecules, such as for example nucleic acids.

The methyl group at the end terminals of the polymer microneedle matrix can determine the effect on the mechanical strength and pliability of the matrix. In some embodiments, the methyl group in the PEG dimethacrylate will provide added fortification at the polymerization site of each PEG molecule and the added fortification will increase the strength of the polymerized matrix, increasing fracture force and force of insertion into or through the skin. Furthermore, a microneedle matrix comprised mainly on end-terminated PEG will be able to swell in size to accommodate extracted or scavenged material. Differential Scanning calorimetry (DSC) analysis (Table 2) shows that there is an increase in T_{g} for the dimethacrylate microneedle matrices, supporting the hypothesis that these needles will have an enhanced mechanical strength and skin piercing.

The majority of the remaining formulation is the functional handle, 2 (dimethylamino)ethyl methacrylate (DMAEM), a chemical that imparts a positive charge to the microneedle matrix. The amino group carries a positive charge that can attract, for example, the negatively charged phosphate backbone of nucleic acids. The last two constituents are important for the fabrication and analysis process: fluorescein o-acrylate is a synthetic, fluorescent dye that will allow for imaging and visualization, and 2,2-diethoxyacetophenone (DEAP) is a photoinitiator needed for the bulk polymerization reaction.

TABLE 2
Glass transition temperature (T_{g}) of four hydrogel
needle matrices via DSC analysis

| Sample Films | First T_{g} ° C. | Second T_{g} ° C. |
|---|---|---|
| PEG₅₇₅diacrylate | −24 | 133 |
| PEG₇₀₀diacrylate | −33 | 202 |
| PEG₅₅₀dimethacrylate | −2 | 214 |
| PEG₇₅₀dimethacrylate | −30 | 254 |

### Fabrication of Cationic Microneedles

The polymer blend matrix was first partially bulk polymerized on a substrate backing of thin, hydrophobic plastic to which a FLUOROCUR (Liquidia Technologies, Inc., North Carolina) was applied and the combination was passed through a hot roll laminator with a pressure of 50 psi. After lamination, the combination was subjected to a final polymerization in a high power UV oven of 10 minutes and the mold was separated from the matrix to produce an array of microneedles. The microneedles were characterized using ESEM (FIG. 17). The tip radii of curvature of these fabricated needles are about 11.97 μm±1.12 (n=15), slightly longer than the intended 10 μm, and the overall height of the individual microneedles is about 384.67 μm±6.38 (n=15). From these ESEM characterizations, the morphology, shape, and structure of these microneedles are provided herein.

### Swelling Study

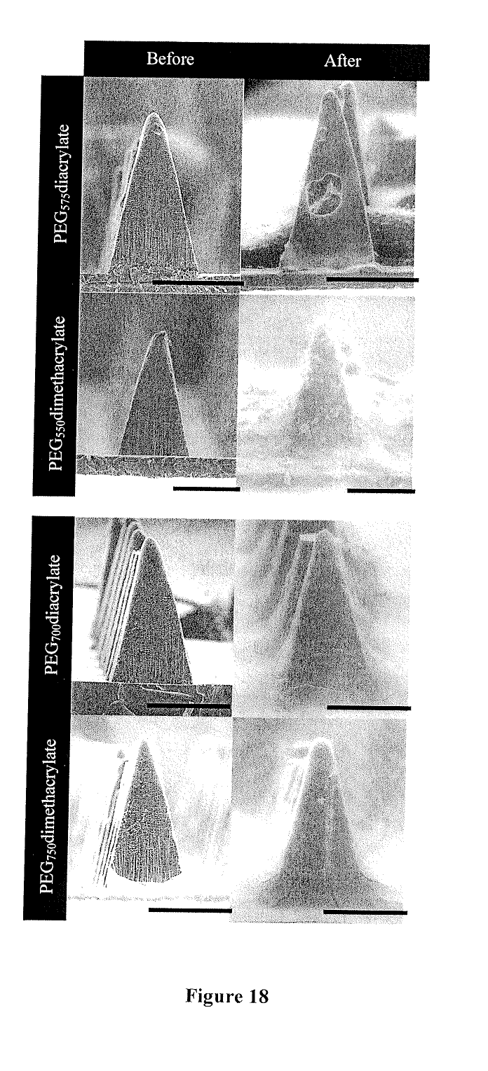

To assess the extent the PRINT microneedle hydrogel matrices could expand to electrostatically attract cytokines when exposed to aqueous solutions and environments, like skin, microneedles of the matrix compositions disclosed herein were swollen to observe morphological changes. Microneedle patches of each hydrogel microneedle matrix (see Table 2) were suspended in water for five minutes; afterward, the microneedle patches were placed in the ESEM at a relative humidity of 60%, similar to the hydration of human skin. After equilibration, the microneedle patches were imaged (FIG. 18). It was seen that the “short” crosslinkers, the PEG₅₇₅diacrylate and PEG₅₅₀dimethacrylate show swelling characteristics that were less desirable than the “long” crosslinkers. The “long” crosslinkers showed swollen microneedles that maintained their shape while still expanding; little visible difference between the acrylate and methacrylate matrices was observed. Therefore, these two matrices were selected to move forward to skin penetration studies to determine the efficacy of each to pierce ex vivo murine tissue.

### Skin Piercing Study

Utilizing the cationic scavenging microneedle array fabrication and composition, the ability of the microneedles to pierce skin in ex vivo murine skin samples was tested. The microneedle patches were rolled on and pressed onto the epidermis of murine skin samples with the gentle force of a thumb. The experimental conditions compared were: a control (no microneedles applied), a hydrogel film (the matrix without any needles), and patches of PEG₇₀₀diacrylate and PEG₇₅₀dimethacrylate. The film and microneedle patches were left in the skin for 10 minutes and removed.

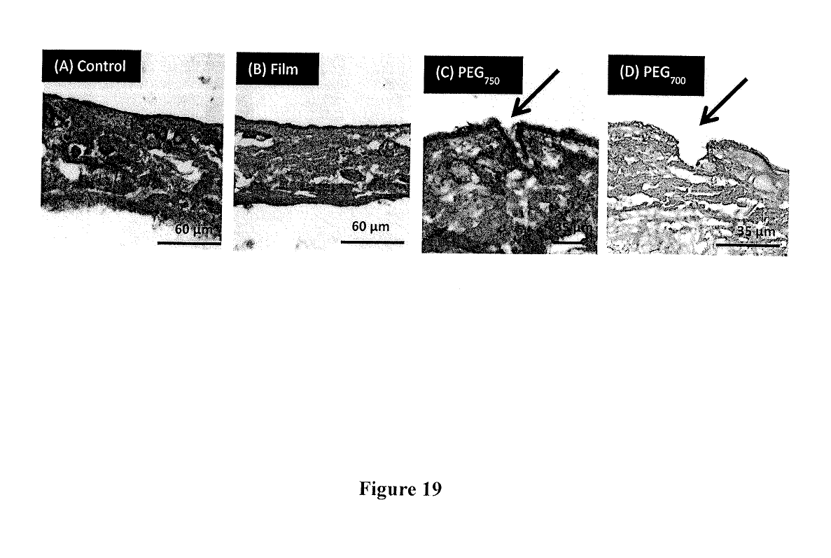

After applying the microneedle patch, a green tissue-staining dye was immediately applied to the skin and subsequently wiped off so that locations of skin penetration could be identified microscopically. Each skin sample was then fixed in a paraformaldehyde fixation solution and stored in a sucrose bath. Tissue samples were then bisected in the z-direction and sectioned on a microtome at 12 μm (−20° C.) for imaging. The tissue samples were then examined with brightfield microscopy to determine the extent of skin penetration. These images, shown in FIG. 19, from sections of murine skin tissue show several sites of epidermal penetration by both varieties of cationic microneedles, while the control and film skin sections did not show these features. In addition, the sites of penetration of the microneedles also show considerable levels of tissue-staining dye, which further exhibits the breach of the epidermal layer. Finally, it was observed that the methacrylate microneedles may penetrate further into the tissue than the acrylate microneedles

1. A system for fabricating a microneedle device, comprising;
depositing a first material on a delivery sheet;
introducing the delivery sheet with deposited first material to a mold;
passing the delivery sheet with deposited first material and mold through a nip-point to introduce at least a portion of first material into cavities in the mold; and
separating the mold from the first material to provide a microneedle device fabricated from the first material with needles mimicking the cavities of the mold.

2. The system of claim 1, further comprising;
before separating, removing the delivery sheet from the first material mated with the mold;
positioning an application layer on the first material mated with the mold;
passing the mold mated with the first material and the application layer through a second nip-point; and
separating the mold from the first material mated with the application layer to provide a microneedle device laminated with the application layer.

3. The system of claim 1, further comprising depositing a second layer onto the first material before introducing the first material to the mold such that the second layer enters the cavities before the first layer.

4. The system of claim 3, wherein the second layer substantially fills the cavities of the mold.

5. The system of claim 3, wherein the second layer includes an active ingredient and the first layer includes a polymer.

6. The system of claim 3, further comprising, controlling a thickness of the second layer deposited on the first layer such that a desired volume of second material enters the cavities of the mold.

7. The system of claim 6, wherein the desired volume includes a desired concentration or dose of second material.

8. The system of claim 1, wherein the cavity includes a tip portion with a diameter smaller than 10 micrometers.

9. The system of claim 1, wherein the cavity includes a tip portion with a diameter smaller than 5 micrometers.

10. The system of claim 1, wherein the cavity includes a tip portion with a diameter smaller than 1 micrometer.

11. The system of claim 1, wherein the modulus of the first material is greater than 2 Gpa.

12. The system of claim 3, wherein the modulus of the combined first material and second material is greater than 2 Gpa.

13. The system of claim 1, wherein the first material includes prefabricated isolated micrometer or nanometer sized particles.

14. The system of claim 3, wherein the first material or the second material includes prefabricated isolated micrometer or nanometer sized particles.

15. The system of claim 1, wherein the system fabricates more than 300 square feet of microneedles per hour.

16. The system of claim 1, wherein the system fabricates more than 500 one square inch microneedle patches per hour.

17. The system of claim 1, wherein the mold is a sheet based system.

18. The system of claim 1, wherein the application layer is bioresorbable.

19. A microneedle device, comprising;
a hydrogel based microneedle device, wherein the hydrogel comprises a PEG diacrylate or PEG dimethacrylate.

20. The microneedle of claim 19, wherein the PEG diacrylate is PEG₇₀₀diacrylate and the PEG dimethacrylate is PEG₇₅₀dimethacrylate.
